(19) United States
(12) Patent Application Publication
(10) Pub. No.: US 2005/0142032 A1
(43) Pub. Date: Jun. 30, 2005

(54) Test system for determining an analyte in a liquid sample

(76) Inventors: Joachim Hoenes, Zwingenberg (DE); Christine Nortmeyer, Mannheim (DE); Carina Horn, Biblis (DE)

Correspondence Address: Roche Diagnostics Corporation, 9115 Hague Road, PO Box 50457, Indianapolis, IN, 46250-0457, US

(21) Appl. No.: 10/977,379

(22) Filed: Oct. 29, 2004

(30) Foreign Application Priority Data
Oct. 31, 2003 (DE) DE 103 50 880.5

(51) Int. Cl. G01N 33/543

(52) U.S. Cl. 422/58; 436/518

(57) ABSTRACT

A test system for determining an analyte in a liquid sample is provided comprising at least two compartments, wherein the detection reactions necessary to determine the analyte are carried out in a first compartment and an analytical determination of at least one substance which participates in the detection reactions and is different from the analyte, the indicator substance, takes place in a second compartment. The two compartments are separated from one another in a manner which allows the indicator substance to pass into the second compartment and at least partially prevents passage of other substances that could interfere with the analytical determination of the indicator substance in the second compartment. At least one other substance is present in an immobilized form in the second compartment which, as a capture substance, selectively enriches the indicator substance in the second compartment. The indicator substance is not a coenzyme and at the same time the capture substance is not a catalytically inactive coenzyme-binding protein.

### BACKGROUND OF THE INVENTION

The present invention is directed to techniques and apparatus employed in medical diagnostics and, more particularly, to a test system for determining analytes in a liquid sample which allows an analyte determination with high specificity and sensitivity, especially when other substances are present in the liquid sample which interfere with the determination of the analyte. The present invention also concerns methods for determining an analyte in a liquid sample and, more particularly, for determining coagulation parameters as well as glycosylated haemoglobin in whole blood or a blood product derived therefrom.

The analytical detection and determination of the concentration of certain biological and medically relevant substances, so-called analytes, in complex samples is an important basis of modern medical diagnostics.

So-called carrier-bound tests are often used for the qualitative and quantitative determination of analytes in liquid samples, in particular in blood or urine. In these dry chemistry methods the reagents are present in a dry form on or in appropriate layers of a solid test carrier which is contacted with the sample. The reaction of liquid sample and reagents results in a detectable signal, especially a change in colour or fluorescence which can be evaluated visually or with the aid of an instrument, typically by means of reflection photometry or fluorimetry. Other detection methods are based on electrochemical methods and for example detect changes in charge, potential or current. Such test carriers are often formed as test strips which are essentially composed of an elongate carrier layer, a detection layer containing the detection reagents and possibly other auxiliary layers, for example filtration layers.

A major problem of these analytical methods is that in many cases the analyte cannot be directly detected in complex sample mixtures since other substances present in the sample liquid apart from the analyte influence or even completely prevent the analyte determination. In such cases a prior separation of the analyte from these interfering substances is necessary in previous methods in order to be able to carry out a specific and sensitive analyte determination at all. Such purification steps prior to the actual analyte determination often require additional steps in the procedure such as washing steps, precipitation reactions, centrifugation steps, adsorption steps, phase separations or filtrations and, hence, such methods are often very time consuming, laborious and require complicated apparatus. Thus, in order to simplify the analysis and make it more economical, it is desirable to avoid such additional purification steps. Another disadvantage of purification steps is that they change the composition of the sample liquid. As a result it is no longer possible to ensure that the analyte is determined in the native reaction environment especially in the case of complex detection reactions involving several reaction steps and reaction partners, for example enzymatic reaction cascades such as blood coagulation, thus falsifying the analyte determination or making it impossible.

In layered test systems a wide variety of blood parameters are often detected by specific enzymatic detection reactions and subsequent detection of a product formed in this process, the so-called indicator substance. However, a prerequisite for this is that the properties of the sample liquid and in particular the presence of other substances in the same sample do not influence the detection of the indicator substance. Thus, a determination of an analyte in blood by optical methods can often not be carried out without a complex pretreatment of the sample since the haemoglobin that is present in a high concentration in blood makes it impossible to optically determine analytes at low concentrations due to its optical properties. Therefore, in these cases a separation of these substances or cells is usually unavoidable in the previously used methods.

A problem which frequently occurs when detecting analytes, especially by means of enzymatic reactions, is that such reaction mixtures often have a very complex composition which, on the one hand, results in a low stability and high susceptibility to interference of the detection reaction. On the other hand, many substances some of which are present at extremely low concentrations in the reaction mixture or whose occurrence or significance is still unknown, are often necessary to allow the detection reaction to occur in an environment which resembles the native environment as closely as possible. An example of this is the coagulation reaction. However, this complex composition is disadvantageous for the detection of the indicator substance in this reaction mixture which is converted during the course of the detection reactions since various interactions can occur between substances present in the sample or in the detection reagent mixture which in turn can have a major effect on the determination of the indicator substance. In each case complicated measures are necessary to separate such interfering components of the liquid sample in order to prevent them from interfering with or preventing the determination of the indicator substance. Especially when determining analytes in whole blood, this means that an additional plasma separation is thus often unavoidable.

An example of this is the determination of blood sugar in whole blood by photometric methods which are based on the principle of an enzymatic conversion of glucose, especially by glucose oxidase or glucose dehydrogenase, as part of a colour generating reaction. In this case, blood cells and especially the red blood corpuscles have to be removed by filtration on the surface of the detection layer so that only an almost colourless plasma can penetrate into the detection layer. The colour generating reaction then takes place in this layer which can be measured photometrically. However, this requires that the red blood corpuscles are reliably separated and that their red colour is held back from the detection layer by additional colour pigments.

### SUMMARY OF THE INVENTION

It is against the above background that the present invention provides certain unobvious advantages and advancements over the prior art. In particular, the inventors have recognized a need for improvements in test system for determining analytes in a liquid sample design.

Although the present invention is not limited to specific advantages or functionality, it is noted that the present invention provides test systems and methods for detecting analytes which enable a sensitive and specific determination of analytes even in complex sample liquids, and satisfies the requirements of an economical and user-friendly routine analysis. This is achieved according to the invention by adding an additional extraction layer to the components of a test system described in the prior art. This additional extraction layer allows the indicator substance used for the analyte determination to be selectively enriched in regions of the test system in which the analytical detection of this indicator substance takes place. The extraction layer according to the invention additionally enables substances to be held back which could interfere with the determination of the indicator substance in the detection layer. Especially in the case of analytes present in blood, the detection reactions for determining the analyte could thus actually take place in whole blood while the indicator particles participating in these reactions are subsequently selectively enriched from this blood compartment in the extraction layer and can then be detected in this layer substantially free of interference. This allows an exact and sensitive determination of analytes in a single test system even without additional separation steps.

In accordance with one embodiment of the present invention, a test system for determining an analyte in a liquid sample is provided comprising at least two compartments, the detection reactions necessary to determine the analyte being carried out in a first compartment, i.e., the reaction space, and an analytical determination of at least one substance involved in the detection reactions, i.e., the indicator substance, being carried out in a second compartment, i.e., the extraction layer. The two compartments are separated from one another in a manner which allows the indicator substance to pass into the second compartment and which at least partially prevents passage of other substances which can interfere with the analytical determination of the indicator substance in the second compartment. Furthermore, at least one other substance is present in an immobilized form in the second compartment which, as a capture substance, selectively enriches the indicator substance in the second compartment. The indicator substance is not a coenzyme and at the same time the capture substance is not a catalytically inactive coenzyme-binding protein.

In accordance with another embodiment of the present invention, a method for determining coagulation parameters in whole blood or a blood product derived therefrom is provided comprising: a) converting a fluorescently labelled thrombin substrate during the course of detection reactions in a first compartment of a test apparatus to form a fluorescent indicator substance; b) enriching an indicator substance by specific capture substances in a second compartment of the test apparatus while excluding interfering sample components; and c) detecting the indicator substance in the second compartment using optical methods.

In accordance with still another embodiment of the present invention, a method for determining glycosylated haemoglobin in whole blood or a blood product derived therefrom is provided comprising: a) providing an indicator substance in a first compartment of a test apparatus, the indicator substance comprising a low molecular weight labelled reagent which specifically binds to glycosylated haemoglobin during the course of a detection reaction; b) enriching the indicator substance that is not bound to glycosylated haemoglobin with specific enriching in a second compartment of the test apparatus, while excluding interfering sample components and excluding the indicator substance bound to glycosylated haemoglobin; and c) detecting the indicator substance in the second compartment using optical methods.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows time courses of mean values of the fluorescence intensity of aminomethylcoumarin in whole blood determined using the test carrier according to the present invention for determining coagulation parameters as a function of the hydroxypropyl-beta-cyclodextrin concentration in the extraction layer; and

FIG. 2 shows time courses of mean values of the fluorescence intensity of aminomethylcoumarin determined using the test carrier according to the present invention for determining coagulation parameters in whole blood of normal donors and Marcumar® patients.

### DETAILED DESCRIPTION OF TYPICAL EMBODIMENTS OF THE INVENTION

In accordance with the present invention, the determination of the analyte does not occur directly but is rather mediated by an indicator substance which is converted or formed during the course of analyte-specific detection reactions. These detection reactions can occur directly in the sample liquid after addition or release of the required detection reagents, typically without an additional sample preparation. The concentration of an indicator substance can be changed in this process either directly from the analyte to be determined or via a more complex chain of reactions which involve the analyte. This indicator substance can have a low molecular weight and can be in an equilibrium distribution between the reaction space and the extraction layer which establishes in particular as a result of diffusion. Capture substances immobilized in the extraction layer selectively enrich the indicator substance in the extraction layer and thus considerably increase the sensitivity of the analyte determination. Hence, analytes which cannot themselves enter into the extraction layer can be determined with high sensitivity. On the other hand, other sample components which could interfere with the determination of the indicator substance such as the strongly absorbing haemoglobin, are substantially excluded from the extraction layer.

The test systems and methods according to the invention can, however, also be used for electrochemical detection methods in which the presence of complex sample mixtures is not such a major problem due to the method of measurement. The enrichment of the indicator substances in the extraction layer as a result of the capture substances that are immobilized there increase the measurement signal which can increase the sensitivity and specificity of the analytical determination.

Test systems in the sense of the present application are all devices which allow a determination of an analyte in a liquid sample. Such test systems typically have a carrier-bound structure. Such test carriers are often in the form of test strips which essentially consist of an elongate support layer made of plastic material and detection elements mounted thereon as test fields. However, test carriers are known which are in the form of small square or rectangular plates. A typical embodiment consists of an inert base support on which an extraction layer according to the present invention is mounted, and which additionally has a reaction space in which the detection reactions involving the analyte and the indicator substance take place, and which thus contains the substances required for the detection reactions. This reaction space can be present as a special layer of the test system or also be formed by the liquid sample itself. In the latter case, the detection reagents can be present in a dry form on the test carrier, especially on or within the extraction layer and are not dissolved and released into the reaction space until the liquid sample is applied which starts the detection reactions in the reaction space. Furthermore, the required detection reagents can be added to the liquid sample in a prior step and subsequently this reaction mixture is applied to the test carrier and, in particular, to the extraction layer.

Analytes which can be determined using test systems or methods according to the present invention can be in the sense of the present application, all particles which are of interest in analytics, especially in clinical diagnostics. In particular, the term “analyte” encompasses atoms, ions, molecules and macromolecules, in particular biological macromolecules such as, for example, nucleic acids, peptides and proteins, lipids, metabolites, cells and cell fragments. Analytes which occur in whole blood and are detected by means of detection reactions which require other substances present in whole blood can be determined particularly advantageously using the methods and test systems according to the invention. Examples of this are analytes involved in blood coagulation such as thrombin or factor Xa which for their determination can employ other substances in the coagulation cascade that are present in whole blood and are often macromolecular and, therefore, such an analyte determination is carried out in whole blood.

Other typical analytes are enzymes or enzyme substrates. Enzymatic analytes may in particular be hydrolases such as, for example, peptidases, esterase, glycosidases, phosphatases and sulfatases. Substrates of these enzymes, in particular natural substrates can also be determined. Competition of the natural substrate with an artificially labelled substrate analogue can be utilized for this purpose. Cleavage of the indicator substances from artificially labelled substrates is lower in the presence of the natural substrates since the cleaving enzyme can in each case only cleave one of the two. This effect can be used to determine the amount of the natural substrates.

Glucose and glycosylated haemoglobin can also be determined as another typical analyte. In this case especially the release of phenylenediamine can be utilized which is an intermediate product of many photometric and electrochemical glucose tests. This phenylenediamine can be used as an indicator substance and for example be concentrated in the extraction layer by cyclodextrins which increases the sensitivity of the detection methods and can avoid the risk of bleeding in the sample.

Other typical analytes may be partners of a specific binding pair. If molecules which bind specifically are allowed to interact with their labelled binding partners in the reaction space and a specific capture substance for the same labelled partner is provided in the extraction layer, an equilibrium distribution establishes between the reaction space and extraction layer such that the more labelled binding partner is present in the layer, the less labelled binding partner is specifically bound in the sample liquid of the reaction space. In this case the label must be selected such that it is suitable for the measurement method and has an adequate affinity for the capture substance. Examples of this are labelled haptens and the specific antibody, labelled cofactors and enzymes or labelled complementary nucleic acids. Also, other binding partners such as biotin/streptavidin or biotin/avidin can be used in this case.

Liquids in the sense of the present invention may be pure liquids and homogeneous or heterogeneous mixtures such as dispersions, emulsions or suspensions. In particular, atoms, ions, molecules and macromolecules, in particular biological macromolecules such as nucleic acids, peptides and proteins, lipids, metabolites and also biological cells or cell fragments may be present in the liquids. Typical liquids of biological origin to be examined are blood, plasma, serum, urine, cerebrospinal fluid, lachrymal fluid, cell suspensions, cell supernatants, cell extracts, tissue lysates, etc. Liquids may, however, also be calibrator solutions, reference solutions, reagent solutions or solutions containing standardized analyte concentrations, so-called standards.

In the present invention the determination or detection of substances means a qualitative as well as a quantitative detection of substances. In particular, it is understood as a determination of the concentration or amount of the respective substance, ascertaining the absence or presence of a substance being also regarded as a determination of the substance.

Analytes are determined using specific detection reactions in a known manner in analytics. The analyte to be determined is not detected directly but rather the analyte to be determined participates in one or more detection reactions in the course of which the indicator substance that is different from the analyte is involved in such a manner that it enables conclusions to be drawn about the concentration of the analyte.

Detection reactions are understood in the present application as chemical and biochemical, in particular enzymatic reactions which involve the analyte to be determined and in the course of which the concentration of an indicator substance that is different from the analyte is changed in a manner which correlates with the concentration of the analyte. The indicator substance can be formed or converted in a single reaction in which the analyte directly participates or, on the other hand, the indicator substance, as in the case of a coagulation measurement, is only formed or converted in the course of a reaction cascade in which the analyte is involved at a different place. Correlation of the amount of converted indicator substance with the amount of analyte contained in the sample is typically based on stoichiometric relationships. A calibration gives a relationship between the measured value for the indicator substance and the concentration of the analyte to be determined. Thus, glucose can for example be converted by glucose oxidase to gluconolactone and hydrogen peroxide in the presence of oxygen, the hydrogen peroxide reacting with a peroxidase in the presence of an indicator to form water and a coloured substance. In the present case this coloured substance corresponds to the indicator substance whose concentration can be determined photometrically or visually. Since the change in the concentration of this indicator substance correlates with the amount of analyte, the determination of this indicator substance can be used to deduce the amount of analyte that is present. Another example of such detection reactions and indicator substances is the enzymatic detection of triglycerides. In this case the sample liquid is firstly mixed with a reaction mixture which contains the substances necessary for the analyte determination in particular enzymes, coenzymes and substrates. The triglycerides are firstly converted by an esterase into glycerol which is in turn converted by a glycerokinase into glycerol-3-phosphate and subsequently by a glutathione peroxidase into dihydroxyacetone phosphate and hydrogen peroxide. This is converted in the presence of a colourless indicator by peroxidase into water and a coloured substance which can be used as an indicator substance for determining the amount of triglycerides. Complex enzymatic detection reaction cascades are also often necessary to determine blood coagulation parameters such as thrombin or factor Xa. One example of a chemical detection reaction is the reaction of glycosylated haemoglobin (HbA_{1c}) with a fluorescent boronic acid as a result of which the amount of free boronic acid decreases with increasing HbA_{1c} concentrations in the sample in correlation with the amount of HbA_{1c}.

In addition to determining the concentration of the analyte itself, the methods according to the invention can also be used to determine the rates of detection reactions and in particular of the conversions of the indicator substance. These values can in turn be used to derive information about the concentrations and activities of the involved substances and in particular the involved enzymes.

An indicator substance in the sense of the present application refers to substances which generate a detectable signal which correlates with the amount or concentration of the analyte. Chromogenic or fluorescent substances can be used especially as indicator substances. Chromogenic substances can form a colour, lose their colour or change their colour. Fluorescent substances can generate fluorescence, lose their fluorescence or change their fluorescent properties. In particular, such an indicator substance can be converted or formed by analyte-specific reactions. Other examples of indicator substances are acids or bases or redox equivalents which can be used especially for electrochemical detection methods.

According to the present invention, the detection reactions proceed in a first compartment, the reaction space, at least up to the formation or conversion of the indicator substance. In addition to the liquid sample, the reaction space contains substances and auxiliary agents necessary to carry out the detection reactions and in particular to form or convert the detection substance. In particular, in the case of enzymatic detection reactions the enzymes, coenzymes, cofactors, substrates, buffer substances, indicators and mediators that are necessary for this are present in the reaction space or are released into the reaction space. This reagent mixture or parts of this mixture can be present in the form of a solution or suspension in an aqueous or non-aqueous liquid or be present as a powder or lyophilisate. In addition, they can be present in the form of a dry chemistry test in which the reagent mixture is applied to a carrier. The carrier can for example be an absorbent or swellable material which is wetted by the sample liquid as a result of which the reagent mixture that is in or on this material in a dry form dissolves in the sample liquid and the detection reactions can take place. The reagent mixture can also be applied to a carrier substrate in the form of soluble layers or films. The detection reactions can be started by contacting the sample liquid and reagent mixture directly in the reaction space or by adding a substance that is essential for the detection reactions at a later time. There are no limitations to the design of this reaction space and it can be adapted to the respective detection method by a person skilled in the field of instrument-based analytics. Thus, for example, the reaction space can be designed as the interior of a reaction vessel such as a cuvette or microtitre plate well. Especially in the case of a dry chemistry test strip, the reaction space can also correspond to the volume of the liquid sample. If, for example, a drop of blood is applied to a test strip provided with detection reagents, it dissolves the reagent mixture which initiates the detection reactions. In this case the drop of liquid itself corresponds to the reaction space. Thus, irrespective of its geometric shape, the reaction space is regarded as the volume in which the detection reactions take place at least up to the conversion or formation of the indicator substance.

According to the present invention, the indicator substance is not detected or determined in the reaction space itself but rather in a special second compartment, the extraction layer. This extraction layer is spatially separated from the reaction space but is in contact with this space in such a manner that liquids and substances dissolved therein that are at least below a certain exclusion size can pass from the reaction space into the extraction layer. According to the present invention, the extraction layer fulfils several functions:
It enables the indicator substance to be detected in a specially suitable environment for this determination.
It can be used to prevent substances that could interfere with the detection of the indicator substance from passing into the extraction layer so that the indicator substance can be detected substantially free of interference.
It contains special substances, so-called capture substances, which selectively enrich the indicator substance in the extraction layer and thus increase the sensitivity and specificity of the analyte determination.

Hence, one of the functions of the extraction layer is to enable the indicator substance to be determined as free of interference as possible. For this it is particularly necessary to create a spatially delimited area in which, on the one hand, the indicator substance is present at a concentration that can be readily detected and correlates with its concentration in the reaction space and in which, on the other hand, interfering substances cannot enter or only to a slight extent so that falsification of the determination of the indicator substance by these interfering substances is substantially avoided.

Substances which can interfere with a determination of the indicator substances are understood in the sense of the present application as all substances or particles which, when present together with the indicator substance in the liquid sample and/or the reagent mixture, impede or prevent a determination of the indicator substance with the respective detection method. Such substances can for example interact specifically or unspecifically with the indicator substance for example as a result of hydrophobic or ionic interactions and thus change the properties of the indicator substances that are utilized in the respective detection method. An example of this is a possible change in the fluorescence properties of indicator substances by interaction with other molecules or by a change in the microenvironment of the indicator substances by such interfering substances which are based on processes such as quenching or fluorescence resonance energy transfer or local changes in the pH. Other such interfering interactions can in particular also occur with proteins present in the sample and are described for example in the “Handbook of Fluorescent Probes and Research Products” (9th Edition, Molecular Probes Europe, Leiden, Netherlands). However, interfering substances can also be substances or particles which, due to their properties, impede or prevent a determination of the indicator substance by at least partially masking the measurement signal of the indicator substance and thus falsify the result of the measurement. In particular, chromogenic substances present in the reaction mixture can result in additional measurement signals in optical detection methods which are superimposed on the signal of the indicator substance such that it is no longer possible to differentiate between the measurement signal of the indicator substance and the effect of interfering substances which impedes or prevents a determination of the indicator substance. Such chromogenic substances can either be already originally present in the sample or only be formed during the course of the detection reactions. For example, analyte determinations by optical detection methods can only be carried out and evaluated to a very limited extent in whole blood since haemoglobin which is present at high concentrations in whole blood absorbs strongly over a wide range of wavelengths and thus often completely masks a specific absorption of the indicator substance which is usually only present in small amounts. There are also similar problems in detection methods based on fluorescent indicator substances since their excitation and emission light is often also almost completely absorbed by the highly concentrated haemoglobin.

In electrochemical detection methods redox substances that interact with the indicator substance can for example also act as interfering substances.

According to the present invention, the extraction layer is spatially separated from the reaction space by designing the extraction layer as a special layer within the overall test system, the extraction layer being a diffusion barrier for certain substances such that substances selectively pass over from the reaction space into the reaction layer. Selective in the sense of the present application does not necessarily mean a complete exclusion of certain substances from a layer or complete passage into a layer. Selective also means that certain substances can selectively enter the layer and can accumulate there or can at least be partially kept out of the layer. Hence, a selective exclusion of a substance by a layer does not necessarily mean the complete absence of this substance in the corresponding compartment but rather only a reduced concentration of this substance in the corresponding area since in practice it is not necessary to have a 100% exclusion of substances.

The extraction layer is separated from the reaction space in particular due to the fact that the extraction layer is a matrix having a selective exclusion size for substances with a molecular weight of more than 1500 g/mol, typically of more than 2000 g/mol, and more typically more than 15000 g/mol. Swellable and absorbent materials that can take up liquid come especially into consideration as such matrix materials. They can for example be corresponding fine-pored fibrous materials such as fleeces, fabrics, knitted fabrics or porous plastic materials. In particular, such matrices can also be non-fibrous materials such as porous or non-porous films, membranes, gel matrices or polymer layers. In a typical embodiment the extraction layer is also in the form of a gel matrix in which the other components of the extraction layer and in particular the capture substances are incorporated. The gel matrix typically has a layer thickness of less than 50 μm, in particular of less than 5 μm and is applied to a support such as an at least partially optically transparent support. The gel matrix is typically a polymer which has a composition based on photopolymerizable monomers such as acrylic monomers, e.g., acrylamide or/and acrylic acid esters, e.g., polyethylene glycol diacrylate or vinyl aromatic monomers, e.g., 4-vinylbenzosulfonic acid, or combinations thereof.

In order to prepare such a gel matrix, a liquid which contains one or more photopolymerizable monomers, capture substances and optionally other additional components of the extraction layer such as colour pigments can, in a typical embodiment, be applied to an at least partially optically transparent support such as a plastic foil and for example be irradiated with UV light from the rear to polymerize the monomers on the support to a predetermined layer thickness. The layer thickness can be controlled by adding absorbing substances to the reagent or/and by means of the duration or intensity of the irradiation. Excess liquid reagent can be removed after the polymerization and used again.

On the other hand the matrix can also be prepared by conventional coating procedures in which a suspension of all reagents required for the extraction layer is applied to a support where it is brought to the required thickness using suitable methods, e.g., with a doctor knife and then dried or completely polymerized.

In addition to covalently cross-linked polymeric gel matrices, it is also possible to use non-covalently cross-linked gels as a component of the extraction layers for example polyelectrolyte gels, e.g., alginate gels cross-linked with divalent ions.

It is also possible to use polyamide, polyvinylidene difluoride, polyethersulfone or polysulfone membranes as matrix materials. The other substances present in the extraction layer and in particular the capture substances can for example be incorporated into the membrane by impregnation. Furthermore, so-called open films can be used for the extraction layer such as those described in EP-B-0 016 387. For this purpose solids are added as fillers in the form of fine, insoluble, organic or inorganic particles to an aqueous dispersion of film-forming organic plastics and the other substances contained in the extraction layer and in particular capture substances are additionally added. Suitable film formers are typically organic plastics such as polyvinyl esters, polyvinyl acetates, polyacrylic esters, polymethacrylic acid, polyacrylamides, polyamides, polystyrenes, mixed polymers of for example butadiene and styrene or of maleic acid ester and vinyl acetate or other film forming, natural and synthetic organic polymers as well as mixtures thereof in the form of aqueous dispersions. The dispersions can be spread on a support to form a uniform layer which results in a water-resistant film after drying. Although the other substances present in the extraction layer and in particular the capture substances are normally added to the dispersion used to prepare the open films, it may also be advantageous to impregnate the formed film with these reagents after its manufacture. It is also possible to pre-impregnate the fillers with the reagents.

The exclusion size of the matrix material can be adjusted in the above-mentioned cases in a manner known to a person skilled in the art by, for example, selecting suitable fillers and concentrations thereof, by controlling the degree of cross-linking of polymeric gel matrices or by a suitable concentration of cross-linking ions in polyelectrolyte gels and in particular be adapted to the interfering substances that are to be excluded.

The indicator substance can be determined in the extraction layer using a wide variety of detection methods known to a person skilled in the field of instrument-based analytics. In particular, it is possible to use optical and electrochemical detection methods. Optical methods, for example, include the measurement of absorption, transmission, circular dichroism, optical rotation dispersion, refractometry or typically fluorescence. Electrochemical methods can in particular be determinations of charge, potential or current. The test systems and methods according to the invention are particularly advantageous for optical methods which suffer particularly from light absorption by interfering substances, in particular blood components.

Immobilized capture substances are an essential component of the extraction layer. Capture substances in the sense of the present application are understood as all substances that interact with the indicator substance and enable this substance to be selectively enriched in the extraction layer. A selective enrichment is understood as an enrichment of the indicator substance which results in a concentration of this substance in the extraction layer that is larger than the concentration that would arise as a result of a purely diffusive flow of this substance into the extraction layer. In particular, the capture substance should selectively enrich the indicator substance in the sense that its concentration relative to the other components of the sample or of the reagent mixture is increased in the extraction layer. For this purpose, the capture substances do not necessarily have to interact substance-specifically with the indicator substance as is for example the case for antigen-antibody interactions. The specificity can also encompass groups of substances that have common properties especially as a result of a similar chemical structure or similar physicochemical properties. For example, all anionic indicator substances can in principle be enriched by cationic capture substances and conversely all cationic indicator substances can be enriched by anionic capture substances. Hence, capture substances can also have a limited specificity for the indicator substance based on a general interaction.

In particular, substances that can specifically enrich indicator substances on the basis of hydrophilic or hydrophobic interactions can be used as capture substances. For example, cyclodextrins or sugar derivatives can be used as capture substances in order to selectively enrich carbohydrates or relatively hydrophobic indicator substances such as aminocoumarins, nitroanilines or phenylenediamines in the extraction layer on the basis of hydrophobic effects. Serum albumins can also be used.

Substances that can specifically enrich indicator substances on the basis of ionic interactions can also be used as capture substances. For example, polyelectrolytes such as polycations or polysulfonic acids can be used as capture substances which, on the basis of electrostatic interactions, selectively enrich oppositely charged indicator substances in the extraction layer.

Furthermore, substances that can specifically enrich indicator substances on the basis for complex-forming properties can be used as capture substances. For example, chelators such as ethylenediamine tetraacetic acid derivatives can be used as capture substances to selectively enrich polyvalent ions in the extraction layer.

Moreover, substances that can specifically enrich indicator substances on the basis of a precipitation reaction can be used as capture substances. For example, anions or cations can be used specifically as capture substances for proteins in that a dissolved protein can be completely or partially deposited as a precipitate in the extraction layer by adding suitable salts.

Furthermore, substances that can enrich indicator substances based on a specific interaction between the partners of a specific binding pair according to the key-lock principle can be used as capture substances. For example, antibodies or antibody fragments can be used specifically as capture substances for special antigens or haptens. In addition, proteins and especially enzymes can be used specifically as capture substances for corresponding cofactors in particular coenzymes, or, in an inactivated form, also for substrates of these enzymes. In addition, nucleic acids and especially DNA or RNA can be used specifically as capture substances for corresponding nucleic acids which hybridize with these nucleic acids and in particular for complementary nucleic acids. Individual partners of other biological or chemical binding pairs such as biotin/streptavidin or biotin/avidin can also be used as capture substances.

According to the present invention, the capture substances are present in an immobilized form in the extraction layer. Immobilization is understood in the sense of the present invention as all processes and measures which have the effect that the capture substances are retained in the extraction layer and cannot reach the reaction space. This is the basis for a selective enrichment of the indicator substances in the extraction layer. In this connection the immobilization can be carried out in various ways. Methods for this are known to a person skilled in the art. It includes in particular the immobilization of the capture substances in the extraction layer. The immobilization can be in particular achieved by binding to a carrier substance which is typically the matrix of the extraction layer. Binding to a carrier can typically take place by adsorption, ion binding or covalent binding. Furthermore, the immobilization can take place by enclosure in a layer in the form of gels, microcapsules or fibres through which the capture substances cannot pass. This immobilizing layer is typically the matrix of the extraction layer. Furthermore, the immobilization can be achieved by cross-linking the capture substances within the extraction layer.

In addition to the capture substances, the extraction layer can contain other substances that can increase the detectability of the indicator substance in this layer. For example, coloured pigments can be added to the extraction layer in order to minimize interference by coloured layers behind it. This can be particularly advantageous in optical detection methods for determining analytes in whole blood in order to minimize the strong background colour of the haemoglobin. For this purpose pigments having a high refractive index can be added to the extraction layer. Titanium dioxide can be typically added in which case particles having an average diameter of about 0.2 to 0.8 μm have proven to be particularly advantageous.

The concentration of the indicator substance in the extraction layer correlates with the concentration of the indicator substance in the reaction space. This concentration of the indicator substance in the reaction space in turn correlates with the concentration of the analyte in the reaction space from which the concentration of the analyte in the liquid sample can in turn be derived when the volume ratios of sample liquid and reaction mixture are known. Thus, a determination of the indicator substance in the extraction layer allows a calculation of the concentration of the analyte in the liquid sample.

The determination of the indicator substance and thus the determination of the analyte can be carried out by a single determination of a measured value, in particular an end point determination or a measurement after a certain time interval or by a measurement over a certain time period in which discrete measured values are determined several times at discrete time intervals or by a measurement over a certain time period with continuous determination of the measured values.

The determination of the analyte or the indicator substance may be followed by other calculations which starting from the result of the analyte determination, yield other derived quantities which in turn can be used as diagnostic parameters. An example of this is the determination of parameters which can give diagnostic information about the presence of a coagulation disorder and are based on a determination of thrombin. Thus, for example, the prothrombin value or Quick value as a parameter for coagulation disorders of the exogenous system can be ascertained from a determination of thrombin and in particular from the time course of thrombin values after adding thromboplastin and calcium to citrated blood. Furthermore, the activated partial thromboplastin time as a parameter for coagulation disorders of the endogenous system can for example be ascertained from a determination of thrombin and in particular from the time course of thrombin values after adding platelet factor III to citrated blood. The activated clotting time is the time in which fresh blood clots in the presence of a contact activator and can for example be determined by determining thrombin and especially from the time course of thrombin values after adding sterile siliceous earth. The activated clotting time in combination with the thrombocyte count can be used to deduce the function of the endogenous coagulation system. Methods for calculating these parameters on the basis of thrombin determinations are known to a person skilled in the field of clinical diagnostics.

The present invention also concerns a test system for determining analytes in a liquid sample which consists of at least two spatially separate compartments wherein the detection reactions, especially involving the indicator substance, necessary to determine the analyte in the liquid sample are carried out in a first compartment, the reaction space, and the analytical determination of the indicator substance is carried out in a second compartment, the extraction layer. In order to increase the specificity and sensitivity, the extraction layer additionally contains, according to the invention, a capture substance in an immobilized form which selectively enriches the indicator substance. According to the present invention, these two compartments are spatially separated in such a manner that in particular passage of the indicator substance into the extraction layer is favoured but, on the other hand, substances which could interfere there with an analytical determination of the indicator substance are at least partially excluded from this layer.

The above-mentioned aspects of the invention can be used either alone or in any combination.

In order that the invention may be more readily understood, reference is made to the following examples, which are intended to illustrate the invention, but not limit the scope thereof.

### EXAMPLES

### Example 1

### Use of the Method According to the Invention to Determine Coagulation Parameters by Means of a Fluorimetric Thrombin Determination

The reaction principle of this thrombin determination is based on the following processes:

Coagulation of a blood sample is for example triggered by adding tissue factor as a result of which the coagulation cascade proceeds and finally prothrombin is converted into thrombin. Pefafluor TH (Pefa-15865; source: Pentapharm Ltd., Engelsgasse 109, CH-4002 Basle) is a specific substrate for thrombin and is proteolytically converted by thrombin with cleavage of aminomethylcoumarin. The fluorescence properties of aminomethylcoumarin are considerably reduced in the substrate form by the amide binding to the tripeptide. The released aminomethyl-coumarin has a strong fluorescence at an excitation wavelength of 342 nm and an emission wavelength of 440 nm and hence it can be used to detect thrombin activity and thus to determine the amount or concentration of thrombin in the sample. The thrombin concentration determined by the release of aminomethylcoumarin correlates with the course of the coagulation cascade. If the thrombin concentration is determined and recorded over a certain period which is usually several minutes after the start of the coagulation reaction, it is possible to determine other coagulation-specific parameters from the time course of the thrombin concentration such as prothrombin time, activated clotting time or activated partial thromboplastin time. These parameters are widely used to diagnose disorders of blood coagulation.

### Example 1a

### Thrombin Determination in Whole Blood and Plasma Without Extraction Layers

Firstly, preliminary experiments were carried out which, in contrast to the method according to the invention, took place without an additional extraction layer. For this, Mowiol 4-88, Hepes buffer, sucrose, glycine, RPLA-4, Pefa-15865, tissue factor, polybrene and mega 8 were added to 5 μl of a non-pretreated whole blood sample. The concentrations of these substances are like those of the sprayed-on coagulation mixture that is described in Example 1c. A clearly visible clotting takes place but no fluorescence signal whatsoever can be detected in the blood. If plasma is used as the sample liquid instead of whole blood, it is possible to unambiguously observe the cleavage of aminomethylcoumarin by fluorescence measurements. This shows that the principle of determining thrombin by cleavage of the strongly fluorescent aminomethylcoumarin from Pefa-15865 can be used to determine coagulation parameters. Furthermore, it was also shown that the simultaneous presence of haemoglobin or erythrocytes make it impossible to carry out such a determination in whole blood since haemoglobin almost completely quenches the fluorescence of aminomethylcoumarin dissolved in the blood. This fluorescence quenching did not occur due to the separation of blood cells and thus also the haemoglobin-containing erythrocytes as part of isolating plasma from whole blood but additional labour-intensive process steps such as centrifugation were necessary which makes the detection method more complex and more susceptible to interference.

### Example 1b

### Aminomethylcoumarin Determination in Extraction Layers Containing Various Capture Substances

A conventional layer structure with filter layers in front to remove interfering substances that is for example used to detect glucose in whole blood cannot be used to measure coagulation by fluorimetric determination of thrombin since the required coagulation factors in the blood of the patient have high molecular weights and would thus be at least partially held back by the layers. Similarly, the phospholipids that are essential for coagulation and which are also very large cannot move in an unimpeded manner. Hence, due to the exclusion criteria of the individual layers in conventional layer structures, an unimpeded coagulation reaction is no longer possible since especially macromolecular coagulation factors cannot pass unhindered into the reaction layer.

Therefore, according to the present invention, test systems and methods were used which operated with the extraction layers described above in order to avoid the disadvantages of prior test systems. In this connection various formulations of extraction layers containing different capture substances that can specifically enrich aminomethylcoumarin in the extraction layer were knife-coated onto a poly-carbonate foil (Pokalon®, 140 μm thickness) which is transparent in the detection area and their fluorescence was measured optically together with blood to which aminomethylcoumarin had been added at a concentration of ca. 30 mg/l. For this purpose the extraction layer was irradiated from below through the transparent carrier with excitation light emitted by an LED. The excitation light excites aminomethylcoumarin to fluoresce and emit fluorescent light. This is measured in a detector after passing a suitable filter which is also located below the transparent carrier.

The basic formulation of the extraction layers have the following composition:

| Substance | Amount | Function |
|---|---|---|
| Mowiol 4-88 solution (20% in | 10 g | Film former |
| water | | |
| PEG wetting agent solution | 10 g | Spreading the sample |
| PEG wetting agent solution | | |
| composed of: | | |
| PEG12000 | 40 g | |
| Geropon T77 solution (0.2%) | 200 g | |
| Transpafill | 2.5 g | Film opening |
| TiO₂ | 1 g | Pigment |

Various amounts of potential capture substances were then admixed with 20 g of this basic formulation. Firstly, mixtures were prepared each containing 5 g polystyrene sulfonic acid, 5 g hydroxypropyl-beta-cyclodextrin, or 5 g PEG 20000 per 20 g of the basic formulation and applied as described above to a polycarbonate foil. Subsequently, whole blood to which aminomethylcoumarin had been added was applied to the respective layers and the fluorescence of the aminomethylcoumarin was determined as described above. The use of an extraction layer containing PEG 20000 in this case only showed a very slight increase in fluorescence of about 1000 relative units within 60 sec after adding the blood sample to the test carrier. The use of an extraction layer containing polystyrenesulfonic acid showed a considerable increase in the fluorescence intensity of about 2500 relative units within 60 sec after adding the blood sample to the test carrier. The use of an extraction layer containing hydroxypropyl-beta-cyclodextrin showed an even more pronounced increase in the fluorescence of more than 3000 relative units within 60 sec after adding the blood sample. Hence, in the following experiments hydroxypropyl-beta-cyclodextrin was used as the capture substance. But it is also possible to use the other said substances as well as further substances as capture substances especially when the sensitivity of the detection is of secondary importance. An increase in the concentration of the aminomethylcoumarin capture substance hydroxypropyl-beta-cyclodextrin from 5 g to 7.5 g results in a further increase in the measured fluorescence. An additional increase to 10 g hydroxypropyl-beta-cyclodextrin only leads to a slight increase in the fluorescence intensity and hence a concentration of 7.5 g hydroxypropyl-beta-cyclo-dextrin per 20 g basic formulation was used in the subsequent experiments.

### Example 1c

### Determination of the Time Course of Coagulation by Means of a Fluorimetric Determination of Thrombin in Whole Blood

On the basis of the preliminary experiments, a layer of 7.5 g hydroxypropyl-beta-cyclodextrin per 20 g basic formulation was applied to a test carrier in the aforementioned manner and sprayed with a coagulation reagent. A test carrier on which a layer was applied which only contained the basic formulation and no additional capture substances served as a control.

This coagulation reagent has the following composition:

| | | | Manu- | | |
|---|---|---|---|---|---|
| Concentration | | Ingredient | facturer | Id. No. | Function |
| 12.5 | g/l | Hepes | Sigma | 706574001 | stabilizing the |
| | | | | | pH |
| 23 | g/l | Glycine | Roche | 2012529-650 | solubility, |
| | | | | | stability |
| 55 | g/l | Sucrose | Roche | 2012391-650 | solubility, |
| | | | | | stability |
| 6.9 | g/l | RPLA | Roche | | protecting |
| | | 4 new | | | protein for TF |
| | | quality | | | |
| 1 | g/l | Mega 8 | Roche | 1813099001 | wetting agent |
| 0.0148 | g/l | Polybrene | Roche | 0435406-958 | heparin |
| | | | (Al- | #1140300 | neutralization |
| | | | drich) | | |
| 1.28 | g/l | Pefa- | Roche | | thrombin |
| (1.9 | mM) | 15865 | | | substrate |
| | | (M = | | | |
| | | 675.79) | | | |
| 218 | μg/l | HrTF | Dade- | 3147487001#11 | activator |
| | | solution | Behring | | |
| | | (tissue | | | |
| | | factor) | | | |

After spraying this reagent onto the coated foils the test carrier was dried (40° C., 10 min).

In order to determine the time course of coagulation, unstabilized blood was cooled in an ice bath until shortly before the start of the measurement and immediately heated to about 35° C. before the measurement. The measurement took place on an unheated measurement block so that the temperature during the determination and the coagulation time course was at about room temperature. Since the coagulation reaction is an enzymatic reaction and is thus strongly temperature dependent, one would therefore expect from the start slower coagulation time courses compared to methods in which the sample is heated to 37° C. The device according to the invention can also be heated by an additional temperature control device which thus also allows more rapid coagulation measurements to be carried out. A person skilled in the field of analytical devices is familiar with such temperature control devices so that the sample liquid can be heated in a simple manner. The fluorescence was measured after adding the sample to the test carrier as previously described.

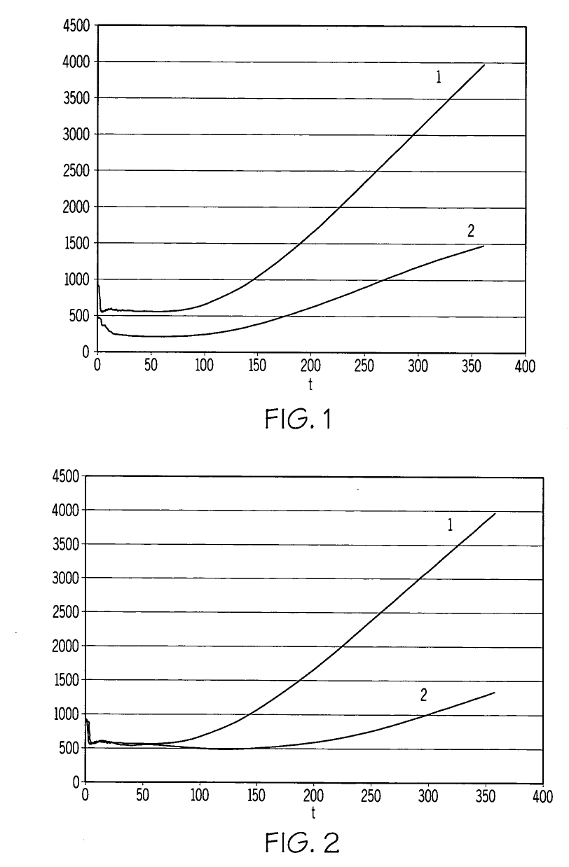

FIG. 1 shows two time courses for the measured fluorescence intensity after addition of whole blood at time t=0 for such test carriers. The time t in seconds is plotted on the abscissa and the measured fluorescence intensity I at an excitation wavelength of 340 nm and an emission wavelength of 470 nm is plotted in relative units on the ordinate. In this case, the upper curve 1 represents a curve of mean values of 11 individual determinations using an extraction layer containing 7.5 g hydroxypropyl-beta-cyclodextrin per 20 g basic formulation and the lower curve 2 is a curve of mean values of 10 control determinations using an extraction layer which only contains the basic formulation and no specific capture substance. It can be clearly seen that the presence of hydroxypropyl-beta-cyclodextrin as a capture substance in the extraction layer can considerably increase the measured fluorescence intensity under otherwise identical test conditions. Thus, with the aid of the present invention, it is in particular possible to utilize a larger measuring range of the measuring instrument resulting in more exact and sensitive measurements. This in turn increases the sensitivity and specificity of analytical methods that are based on such measurements.

### Example 1d

### Comparison of the Time Course of Coagulation by Means of a Fluorimetric Determination of Thrombin in Whole Blood of Normal Donors and Marcumar® Patients

Marcumar® (Hoffmann-La Roche Aktiengesellschaft, Basle, Switzerland) is an orally administered coagulation inhibitor which delays but does not completely abolish blood coagulation. Marcumar® inhibits the vitamin K-dependent synthesis of the coagulation factors II, VII, IX and X. Marcumar® is primarily used for the prevention and treatment of venous thrombosis, myocardial infarctions and pulmonary embolisms. Since its action is associated with an elevated bleeding tendency, monitoring by regular control measurements is absolutely essential in order that the dosage can be adjusted if necessary. The monitoring is based on regular determinations of coagulation-specific parameters carried out by the doctor or the patients themselves. The method according to the invention can be typically used for this purpose.

FIG. 2 shows two time courses of the measured fluorescence intensity after adding whole blood from normal donors and Marcumar® patients at time t=0. The time t in seconds is plotted on the abscissa and the measured fluorescence intensity I at an excitation wavelength of 340 nm and an emission wavelength of 470 nm is plotted in relative units on the ordinate. In both cases, extraction layers containing 7.5 g hydroxypropyl-beta-cyclodextrin per 20 g basic formulation were used. The upper curve 1 represents a curve of mean values of 12 measurements using whole blood from normal donors as the sample liquid and the lower curve 2 is a curve of mean values of 12 control measurements using whole blood from Marcumar® patients as the sample liquid. This clearly shows that the different time courses between normal donors and Marcumar® patients can be recorded with the method according to the invention. Whereas with healthy normal donors a considerable increase in the fluorescence intensity from 550 relative units to about 4000 relative units after 360 seconds is observed about 60 seconds after sample application, in Marcumar® patients there is firstly even a slight decrease in the measured fluorescence intensity and only after ca. 120 seconds is a slight increase in fluorescence intensity to ca. 1300 relative units after 360 seconds observed. This is due to the delayed coagulation caused by the Marcumar® medication.

The principle of enzymatic cleavage of an indicator substance is independent of the cleaved species and the respective substrate. Other substrates, indicator substances and capture substances can also be used.

This example clearly shows that the method and test systems according to the present invention enable a sensitive and specific determination of the concentration of an analyte in a complex sample mixture. Furthermore, the method and test systems according to the present invention enable the concentration of the analyte to be continuously determined over a certain time period or at discrete time intervals without interfering with the course of the detection reactions.

### Example 2

### Determination of Glycosylated Haemoglobin by Means of Fluorescently Labelled Boronic Acids

The principle according to the present invention of determining analytes based on the determination of an indicator substance in a special extraction layer in which the indicator substance is enriched by means of special capture substances and interfering substances remain exclusively in the extraction layer, can be applied to other analyte and parameters.

Another example is the determination of glycosylated haemoglobin (HbA_{1c}) by means of fluorescently labelled boronic acids. HbA_{1c} is a haemoglobin to which glucose is covalently bound. HbA_{1c} also occurs in small amounts in the erythrocytes of healthy persons but occurs to an increasing extent in diabetics depending on the long-term blood sugar level. Hence, HbA_{1c} is especially suitable for retrospectively assessing the carbohydrate balance of these patients commensurate with the average lifespan of erythrocytes of 3 to 4 months and, as a long-term parameter, supplements the monitoring of blood sugar levels in addition to the short-term parameter blood glucose content. In this context the determination of HbA_{1c} is of major diagnostic importance.

Fluorescently labelled boronic acids can be used especially as an indicator substance and special sugars and sugar derivatives, in particular diols, can be used as corresponding capture substances in the extraction layer.

One embodiment of a test carrier according to the present invention for determining HbA_{1c} in whole blood comprises a test carrier on which an extraction layer is applied which for example contains cis-diols, e.g., polysugars or polyalcohols, in an immobilized form as a specific capture substance. In addition, a reagent mixture which contains at least one lysis reagent such as saponins and a certain amount of the fluorescently labelled boronic acid which is for example labelled with aminomethylcoumarin or pyrene trisulfonate is applied to the test carrier and typically directly on the surface of the previously applied extraction layer. After applying the liquid sample, in this case whole blood, the reagent mixture is dissolved and the detection reaction is started in the reaction space which in this case corresponds to the volume of the liquid sample. In this process the boron group of the fluorescently labelled boronic acid binds covalently to the glucose units of HbA_{1c} which is present in the liquid sample. The amount of fluorescently labelled boronic acid bound to haemoglobin correlates with the concentration of HbA_{1c} in the sample. The more HbA_{1c} is present in the sample, the more fluorescently labelled boronic acid is bound to haemoglobin and the lower is the concentration of free fluorescently labelled boronic acid in the reaction space. Since the free fluorescently labelled boronic acid serves as an indicator substance, this example shows that indicator substances do not necessarily have to be formed in the reaction space as part of the detection reactions but rather that a conversion of indicator substances that are already present can also be used for the analyte determination. The extraction layer has an exclusion size which does not allow haemoglobin to penetrate into it since haemoglobin would prevent the fluorimetric detection of the fluorescently labelled boronic acids. Hence, the fluorescently labelled boronic acids bound to haemoglobin cannot penetrate into the detection layer. Instead, the free fluorescently labelled boronic acids are used as indicator substances since these can enter the extraction layer due to their considerably smaller molecular size and, due to their specific properties, accumulate there due to their interaction with the special capture substances. The more HbA_{1c} is present in the sample the less free fluorescently labelled boronic acid can enter the extraction layer and the smaller is the measured signal. The fluorescently labelled boronic acids are detected in the extraction layer with the aid of optical fluorescence methods. The diagnostic suitability of such a test system according to the invention can be further increased by designing it as a multiparameter test system. Thus, for example, several different analytes which are known to exhibit a characteristic change in concentration or are generally absent or present when a certain clinical picture is present, can be determined in a common test system. The simultaneous measurement of several such related parameters enables the determination of different analytes at the same time and in a single operation which yields rapid diagnostic information. Furthermore, by taking into consideration specific combinations of the individual analytical results, it is often possible to obtain diagnostic information which would not be possible by only considering one parameter. Especially, the specificity and/or sensitivity of the diagnostic method can be increased in this manner. In the present case a HbA_{1c} determination can in particular be combined with the determination of the total haemoglobin content in the blood sample. These two measurements can subsequently be related to one another. In particular, the HbA_{1c} proportion of the total haemoglobin content can be determined so that more exact diagnostic information is possible on the basis of this common approach.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

1. A test system for determining an analyte in a liquid sample comprising at least two compartments, the detection reactions necessary to determine the analyte being carried out in a first compartment and an analytical determination of at least one indicator substance being carried out in a second compartment, wherein
a) the two compartments are separated from one another in a manner which allows the indicator substance to pass into the second compartment and which at least partially prevents passage of other substances which can interfere with the analytical determination of the indicator substance in the second compartment;
b) at least one other substance is present in an immobilized form in the second compartment which, as a capture substance, selectively enriches the indicator substance in the second compartment; and
c) the indicator substance is not a coenzyme and at the same time the capture substance is not a catalytically inactive coenzyme-binding protein.

2. The test system of claim 1, wherein the indicator substance participates in the detection reaction and is different from the analyte.

3. The test system of claim 1, wherein the liquid sample comprises a biological sample.

4. The test system of claim 3, wherein the biological sample comprises whole blood or a blood product derived therefrom.

5. The test system of claim 4, wherein the blood product comprises serum or plasma.

6. The test system of claim 1, wherein a chemical or enzymatic detection reaction for determining the analyte takes place in the first compartment in which the indicator substance is formed or converted in a manner which correlates with the presence of the analyte in the liquid sample.

7. The test system of claim 6, wherein the indicator substance is formed or converted in a manner which correlates with the concentration of the analyte in the liquid sample.

8. The test system of claim 7, wherein the indicator substance is formed or converted in a manner which correlates with the concentration of the analyte in the liquid sample on the basis of stoichiometric relationships.

9. The test system of claim 1, wherein the indicator substance has a molecular weight of less than about 15,000 g/mol.

10. The test system of claim 1, wherein the indicator substance has a molecular weight of less than about 2,000 g/mol.

11. The test system of claim 1, wherein the indicator substance has a molecular weight of less than about 1,500 g/mol.

12. The test system of claim 1, wherein the indicator substance establishes an equilibrium distribution between the first and second compartment.

13. The test system of claim 12, wherein the equilibrium distribution between the first and second compartment is established by diffusion.

14. The test system of claim 1, wherein the two compartments are separated by designing the second compartment in the form of a selectively permeable matrix.

15. The test system of claim 14, wherein the selectively permeable matrix is selected from a gel, film, membrane, or polymer layer.

16. The test system of claim 1, wherein the selective enrichment of the indicator substance in the second compartment is based on specific interactions between the indicator substance and the capture substance.

17. The test system of claim 16, wherein the selective enrichment of the indicator substance in the second compartment is further based on a) hydrophilic/hydrophobic interaction; b) ionic interaction; c) complex formation; d) a chemical precipitation reaction; e) specific binding between the partners of a specific binding pair; or f) combinations thereof.

18. The test system of claim 17, wherein the complex formation comprises chelate formation.

19. The test system of claim 17, wherein the specific binding between the partners of a specific binding pair are between the partners of a binding pair according to the lock and key principle selected from antibodies/antigens, proteins/cofactors, complementary nucleic acids, biological binding pairs, or combinations thereof.

20. The test system of claim 19, wherein the biological binding pairs are biotin/avidin or biotin/streptavidin.

21. The test system of claim 1, wherein the capture substance in the second compartment is selected from a carbohydrate, a polyelectrolyte, a complexing agent, an anion, a cation, a binding partner of a specific binding pair, or combinations thereof.

22. The test system of claim 2.1, wherein the carbohydrate is selected from cyclodextrin, polyethylene glycol, albumin, or combinations thereof.

23. The test system of claim 21, wherein the polyelectrolyte is selected from polysulfonic acid, a polycation, or combinations thereof.

24. The test system of claim 21, wherein the complexing agent comprises an ethylenediamine tetraaceitc acid derivative.

25. The test system of claim 21, wherein the binding partner of a specific binding pair is selected from antibodies/antigens, proteins/cofactors, complementary nucleic acids, biological binding pairs, or combinations thereof.

26. The test system of claim 25, wherein the biological binding pairs are biotin/avidin or biotin/streptavidin.

27. The test system of claim 1, wherein the capture substance is immobilized by enclosure in a matrix.

28. The test system of claim 27, wherein the matrix which immobilizes the capture substance at the same time separates the two compartments.

29. The test system of claim 1, wherein the indicator substance is determined in the second compartment by optical or electrochemical methods.

30. The test system of claim 29, wherein the optical method comprises fluorimetric or photometric methods.

31. The test system of claim 29, wherein the electrochemical method comprises amperometric or potentiometric methods.

32. A method for determining coagulation parameters in whole blood or a blood product derived therefrom comprising:
a) converting a fluorescently labelled thrombin substrate during the course of detection reactions in a first compartment of a test apparatus to form a fluorescent indicator substance;
b) enriching the indicator substance by specific capture substances in a second compartment of the test apparatus while excluding interfering sample components; and
c) detecting the indicator substance in the second compartment using optical methods.

33. The method of claim 32 further comprising determining thrombin content and parameters derived therefrom selected from prothrombin time, activated clotting time, and activated partial thromboplastin time.

34. The method of claim 32, wherein the fluorescently labelled thrombin substrate is Pefafluor TH.

35. The method of claim 32, wherein the fluorescent indicator substance is aminomethylcoumarin.

36. The method of claim 32, wherein the specific capture substrate is selected from hydroxypropyl-beta-cyclodextrin or polyethylene glycol 20000.

37. The method of claim 32, wherein the second compartment is in the form of an open film matrix.

38. The method of claim 32, wherein the interfering sample components are blood cells and chromophoric substances.

39. The method of claim 38, wherein the chomophoric substance is haemoglobin.

40. The method of claim 32, wherein the optical method is a fluorescent-optical method.

41. A method for determining glycosylated haemoglobin in whole blood or a blood product derived therefrom comprising:
a) providing an indicator substance in a first compartment of a test apparatus, the indicator substance comprising a low molecular weight labelled reagent which specifically binds to glycosylated haemoglobin during the course of a detection reaction;
b) enriching the indicator substance that is not bound to glycosylated haemoglobin with specific enriching in a second compartment of the test apparatus, while excluding interfering sample components and excluding the indicator substance bound to glycosylated haemoglobin; and
c) detecting the indicator substance in the second compartment using optical methods.

42. The method of claim 41, wherein the low molecular weight labelled reagent is a low molecular weight fluorescently labelled boronic acid.

43. The method of claim 41, wherein the specific capture substances are selected from carbohydrates and diols.

44. The method of claim 41, wherein the interfering sample components are blood cells and chromophoric substances.

45. The method of claim 44, wherein the chromophoric substance is haemoglobin.

46. The method of claim 41, wherein the optical method is a fluorescent optical method.
